(19) United States
(12) Patent Application Publication
(10) Pub. No.: US 2025/0134834 A1
(43) Pub. Date: May 1, 2025

(54) RETINOL FORMULATION (IV)

(71) Applicant: DSM IP Assets B.V., Heerlen (NL)

(72) Inventors: Linda HAENDEL, Kaiseraugst (CH); Peter Louis HOUSTON, Kaiseraugst (CH); Eileen JACKSON, Kaiseraugst (CH); Lise Anne KOHLER, Kaiseraugst (CH); Christine MENDROK-EDINGER, Kaiseraugst (CH); Fabiola PORTA, Kaiseraugst (CH); Pascal Christian SIEBER, Kaiseraugst (CH); Christos TSEKOU, Kaiseraugst (CH); Angela WILDERMANN, Kaiseraugst (CH)

(21) Appl. No.: 18/838,710

(22) PCT Filed: Feb. 21, 2023
(86) PCT No.: PCT/EP2023/054256

§ 371 Date: Aug. 15, 2024

Related U.S. Application Data
(60) Provisional application No. 63/312,478, filed on Feb. 22, 2022.

(30) Foreign Application Priority Data
Apr. 13, 2022 (EP) 22168060.6
May 5, 2022 (EP) 22171756.4

(51) Int. Cl. A61K 31/07; A61K 8/37; A61K 8/67; A61K 47/14; A61K 47/22; A61K 47/24; A61K 47/44; A61Q 19/00

(52) U.S. Cl. CPC A61K 31/07; A61K 8/375; A61K 8/671; A61K 8/678; A61K 47/14; A61K 47/22; A61K 47/24; A61K 47/44; A61Q 19/00; A61K 2800/522; A61K 2800/85

(57) ABSTRACT

The present invention relates to a new formulation, which comprises a high amount of retinol, which has a defined mixture of cis and trans isomers, in a specific solvent and in the presence of mixed tocopherol.

The present invention relates to a new formulation, which comprises a high amount of retinol, which has a defined mixture of cis and trans isomers, in a specific solvent and in the presence of mixed tocopherol.

Retinol, which is compound of the following formula

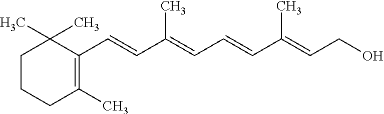 [SMILES: CC1=C(/C=C/C(C)=C/C=C/C(C)=C/CO)C(C)(C)CCC1]

(without any indication of the stereochemistry) is a compound with very interesting prop-erties in a variety of fields of applications.

Next to the application in food, feed, and pharmaceutical, it is also very useful in personal care applications.

When used in the field of personal care it is mainly used for the maintenance of the skin.

Applying retinol topically it includes the following benefits:
Preventing wrinkles due to its minimizing effect, as well as smoothing out existing fine lines and wrinkles.
Brightening dull skin by exfoliating at a cellular level, which results in brighter and smoother new skin.
Regulating oily skin and minimizing breakouts.
Fading dark age spots, sun spots and hyperpigmentation and evening out com-plexion over time.

To produce end-market products (like creams etc), it is necessary to provide a formulation comprising retinol, which can be incorporated into the end market product.

It is very advantageous that the formulation, which is used to produce the end-market product, comprises the retinol in a high amount, which means that not so much of solvent and other ingredients are present. This means that the concentration of such solvents and other ingredients in the end market product can kept low and the formulation of the present invention can be used in a wide range of applications.

Furthermore, such a formulation with a high amount of retinol needs to be stable so that the content of retinol is not decreasing during the storage of the formulation.

Furthermore, it was also a goal not to use antioxidants such as butylated hydroxytoluene (BHT) or butylated hydroxyanisole (BHA), because they are banned in a variety of coun-tries for specific applications.

It was found that when a specific solvent (for the retinol) and mixed tocopherols (as anti-oxidant) were chosen then it was possible to produce a high concentrated and stable retinol formulation. The retinol used in the formulation according to the present invention has a defined cis/trans ratio, which is stable during storage.

The choice of the specific solvent is crucial for the formulation according to the present invention. The solvent used in the formulation is a compound of formula (I)

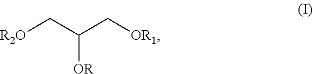

wherein
R is a

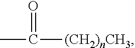

wherein n is an integer from 4 to 16;

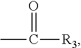

wherein R₃ is a C₁₄-C₂₂ alkene group, and
R₁ is a

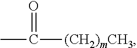

wherein m is an integer from 4 to 16, and
R₂ is a

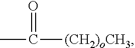

wherein o is an integer from 4 to 16; or

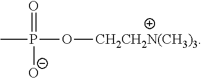

The present invention relates to a formulation (F) comprising
40-75 weight-% (wt-%), based on the total weight of the formulation, of retinol, and
20-55 wt-%, based on the total weight of the formulation, of at least one solvent, and
0.1-5 wt-%, based on the total weight of the formulation, of mixed tocopherol,
wherein the at least one solvent is a compound of formula (I)

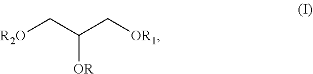

wherein
R is a

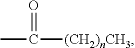

wherein n is an integer from 4 to 16;

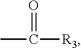

wherein R₃ is a C₁₄-C₂₂ alkene group, and
R₁ is a

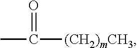

wherein m is an integer from 4 to 16, and
R₂ is a

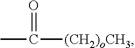

wherein o is an integer from 4 to 16; or

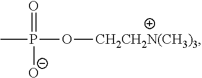

and wherein
the retinol is a mixture of cis and trans isomers wherein
the cis/trans ratio within said retinol mixture is less than 0.01 (1:110), a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.003.

Furthermore, the formulation according to the present invention can also comprise up to 2 wt-% (preferably 0.05 to 1.5 wt-%), based on the total weight of the formulation, of sunflower oil.

The present invention relates to a formulation (Fₐ), which is formulation (F), wherein the formulation comprises up to 2 wt-% (preferably 0.05 to 1.5 wt-%), based on the total weight of the formulation, of sunflower oil.

The present invention relates to a formulation (F1) consisting of
40-75 wt-%, based on the total weight of the formulation, of retinol, and
20-55 wt-%, based on the total weight of the formulation, of at least one solvent, and
0.1-5 wt-%, based on the total weight of the formulation, of mixed tocopherol,
wherein the at least one solvent is a compound of formula (I)

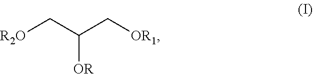

wherein
R is a

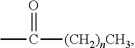

wherein n is an integer from 4 to 16;

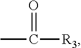

wherein R₃ is a C₁₄-C₂₂ alkene group, and
R₁ is a

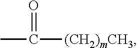

wherein m is an integer from 4 to 16, and
R₂ is a

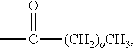

wherein o is an integer from 4 to 16; or

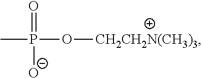

and wherein
the retinol is a mixture of cis and trans isomers wherein
the cis/trans ratio within said retinol mixture is less than 0.01 (1:110), a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.003.

The present invention relates to a formulation (Fₐ1) consisting of
40-75 wt-%, based on the total weight of the formulation, of retinol, and
20-55 wt-%, based on the total weight of the formulation, of at least one solvent, and
0.1-5 wt-%, based on the total weight of the formulation, of mixed tocopherol, and
up to 2 wt-% (preferably 0.05-1.5 wt-%), based on the total weight of the formulation, of sunflower oil,
wherein the at least one solvent is a compound of formula (I)

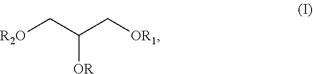

wherein
R is a

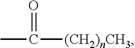

wherein n is an integer from 4 to 16;

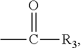

wherein R₃ is a C₁₄-C₂₂ alkene group, and
R₁ is a

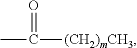

wherein m is an integer from 4 to 16, and
R₂ is a

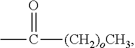

wherein o is an integer from 4 to 16; or

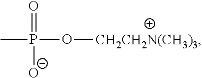

and wherein
the retinol is a mixture of cis and trans isomers wherein
the cis/trans ratio within said retinol mixture is less than 0.01 (1:110), a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.003.

The present invention relates to a formulation (F2) consisting essentially of
40-75 wt-%, based on the total weight of the formulation, of retinol, and
20-55 wt-%, based on the total weight of the formulation, of at least one solvent, and
0.1-5 wt-%, based on the total weight of the formulation, of mixed tocopherol,
wherein the at least one solvent is a compound of formula (I)

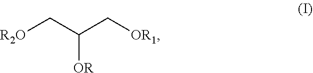

wherein
R is a

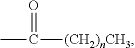

wherein n is an integer from 4 to 16;

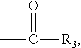

wherein R₃ is a C₁₄-C₂₂ alkene group, and
R₁ is a

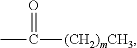

wherein m is an integer from 4 to 16, and
R₂ is a

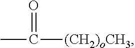

wherein o is an integer from 4 to 16; or

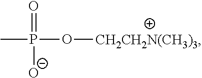

and wherein
the retinol is a mixture of cis and trans isomers wherein
the cis/trans ratio within said retinol mixture is less than 0.01 (1:110), a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.003.

The present invention relates to a formulation (Fₐ2) consisting essentially of
40-75 wt-%, based on the total weight of the formulation, of retinol, and
20-55 wt-%, based on the total weight of the formulation, of at least one solvent, and
0.1-5 wt-%, based on the total weight of the formulation, of mixed tocopherol, and
up to 2 wt-% (preferably 0.05-1.5 wt-%), based on the total weight of the formulation, of sunflower oil,
wherein the at least one solvent is a compound of formula (I)

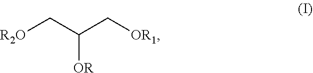

wherein
R is a

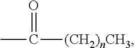

wherein n is an integer from 4 to 16;

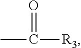

wherein R₃ is a C₁₄-C₂₂ alkene group, and
R₁ is a

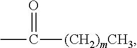

wherein m is an integer from 4 to 16, and
R₂ is a

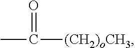

wherein o is an integer from 4 to 16; or

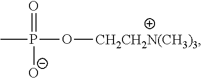

and wherein
the retinol is a mixture of cis and trans isomers wherein
the cis/trans ratio within said retinol mixture is less than 0.01 (1:110), a cis/trans ratio of 0.0099, 0.0095, 0.009, 0.0085, 0.008, 0.0075, 0.007, 0.0065, 0.006, 0.0055, 0.005, 0.0049, 0.0045, 0.0043, 0.004, 0.0035, 0.003, 0.0028, 0.0025, 0.0022, 0.002, 0.0018, 0.0015, 0.001, 0.0005 or less, such as 0.0001 or less, more preferably ranges of 0.0099 to 0.0001, 0.008 to 0.001, 0.007 to 0.002, 0.0099 to 0.001, 0.005 to 0.001, 0.005 to 0.0001, 0.005 to 0.0005, 0.006 to 0.001, 0.009 to 0.0001, 0.008 to 0.0001, 0.006 to 0.0001, 0.0055 to 0.0001, 0.005 to 0.002, 0.007 to 0.0001, 0.0045 to 0.0001, 0.004 to 0.0001, 0.0035 to 0.0001, 0.003 to 0.0001, 0.005 to 0.0025, 0.004 to 0.0005, 0.006 to 0.0005, 0.006 to 0.0008, 0.006 to 0.0015, 0.006 to 0.002, 0.006 to 0.003, 0.006 to 0.0004, 0.006 to 0.0025, most preferably a cis/trans ratio of 0.003.

The cis/trans ratio of the retinol mixture as given herein refers to the wt-% ratio of the respective all trans isomer to the sum of all cis-isomers of retinol as determined by known methods such as e.g. HPLC, assuming the same response factor for all isomers.

The formulation according to the present invention is no emulsion. The formulation according to the present invention is an oil formulation. This means that the water content of the inventive formulation can be kept as low as possible. No water is added to the formulation intentionally. It might be possible that the ingredients of the formulation according to the present invention can contain traces of water.

The oil formulation of retinol according to the present invention using the solvents of the present invention as solubilizer is ensuring an easy and more flexible use of such a solu-tion in further applications, while an emulsification route (having more ingredients) of such active would have detrimental effect on final applications.

The present invention relates to a formulation (F′), which is formulation (F) or (Fₐ), wherein the formulation comprises less than 2 wt-%, based on the total weight of the formulation, of water.

The present invention relates to a formulation (F″), which is formulation (F) or (Fₐ), wherein the formulation comprises less than 1 wt-%, based on the total weight of the formulation, of water.

The present invention relates to a formulation (F″′), which is formulation (F) or (Fₐ), wherein the formulation comprises less than 0.5 wt-%, based on the total weight of the formulation, of water.

The mixture of cis and trans isomers of retinol used in the formulations according to the present invention can either be prepared by admixing the respective all trans isomer with one or more cis isomers obtained by chemical or biological processes. Methods to prepare such all trans and/or cis isomers are well known to a person skilled in the art.

Alternatively, the mixture can be prepared in said isomer ratio by adjusting the processes accordingly.

Advantageously, in all embodiments of the present invention, the retinol used in the formulations according to the present invention, is biologically produced through a fermentation process, wherein trans-retinol produced by fermentation can be treated with heat to form cis-retinol, to achieve the proper levels mentioned in all the embodiments of the present invention invention (see e.g. McBee et al., JBC, Vol. 276, No. 51, pp. 48483-48493, 2001).

Advantageously, in all embodiments of the present invention, the mixture of cis and trans isomers of retinol, with a ratio of cis-retinol to trans-retinol of less than 1:100 (i.e. a ratio of less than 0.01), particularly mixtures of retinol isomers with a cis/trans ratio of about 0.003, preferably wherein said retinoids as defined herein are biologically produced through a fermentation process, have a yellowish color.

As known in the art, color can be precisely described in several different co-ordinate systems, such as XYZ, RGB, CYMK, or L*a*b* (CIELAB according to EN ISO/CIE 11664-4:2019). A preferred method is the definition via the L*a*b* system. The skilled person knows which instrument to use depending on the different color measurement systems and how to measure the color of the mixtures as described herein.

A “yellowish” color as used herein means a color as defined by the L*a*b* color system, particularly wherein L*a*b* being (3<L*<100, −25<a*<30,10<b*<150), such as e.g. L* being in the range of 50 to 100, a* being in the range of −25 to 10 and b* being in the range of 40 to 150, preferably wherein L* being in the range of 80 to 100, a* being in the range of −22 to 1, b* being in the range of 40 to 85 or 100 to 140, more preferably L* being in the range of 80 to 95, a* being in the range of −17 to −1, b* being in the range of 105 to 135, particularly L* being about 80, 81, 82, 83, 84, 85, 86, 87, 88, 89, 90, 91, 92, 93, 94, 95, 96, 97, 98, 99, 100 and a* being about −24.9, −24, −23, −22, −21, −20, −19, −18, −17, −18, −17, −16, −15, −14, −13, −12, −11, −10, −9, −8, −7, −6, −5, −4, −3, −2, −1, 0 and b* being about 40, 41, 42, 43, 44, 45, 46, 47, 48, 49, 50, 51, 52, 53, 54, 44, 56, 57, 58, 59, 60, 61, 62, 63, 64, 65, 66, 67, 68, 69, 70, 71, 72, 73, 74, 75, 76, 77, 78, 79, 80, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135.

In a particular embodiment, the mixture of cis and trans isomers of retinol used in the formulations according to the present invention, are produced in a fermentation process using suitable retinol producing host cells, such as e.g. bacterial or fungal cells, (see e.g. Sun et al, ACS Synth. Biol. 2019 Sep. 20;8 (9): 2131-2140; Jang et al., Microbial Cell Fac-tories 2011, 10:59), wherein the cells are expressing the respective enzymes, such as e.g. trans-selective enzymes, i.e. beta-carotene oxidase (BCOs) involved in biosynthesis of retinol from conversion of beta-carotene into retinal, that might be further enzymatically converted into retinol. The fermentation is fed ethanol, corn sugar or corn oil all derived from agricultural production. The fermentation products comprising retinol might be ex-tracted in an aliphatic phase and subsequently purified to crystalline forms.

The present invention relates to a formulation (F3), which is formulation (F), (Fₐ), (F′), (F″), (F″′), (F1), (Fₐ1), (F2) or (Fₐ2), wherein the retinol used in the formulation is biologically produced through a fermentation process.

The term “biologically produced” as used herein means that the retinol is produced by the help of biotechnological process, such as a fermentation process including cultivation of a suitable (carotenoid and/or retinoid producing) host cell expressing the respective enzymes involved in conversion of a suitable carbon source into retinal and furthermore into retinol as defined herein, wherein the host cell might be selected from bacteria, fungi, particularly yeast, plant, or algae. “Bio-produced”, “biologically-derived” and “biologically produced” are used synonymously herein. Accordingly, said biologically produced retinol is composed of carbon from atmospheric carbon dioxide (also referred to as carbon of atmospheric origin) converted to sugars and starches by green plants. It also includes the use of isolated and/or immobilized enzymes in a process for generation of the retinol mixture as defined herein, such as specific enzymes capable of selectively catalyzing the formation of the specific trans/cis ratio of retinol in a mixture as defined herein.

“Carbon of atmospheric origin” as used herein refers to carbon atoms from carbon dioxide molecules that have recently, in the last few decades, been free in the earth's atmosphere. Such carbons in mass are identifiable by the presence of particular radioisotopes as described herein. “Green carbon”, “atmospheric carbon”, “environmentally friendly carbon”, “life-cycle carbon”, “non-fossil fuel-based carbon”, “non-petroleum-based carbon”, “carbon of atmospheric origin”, and “biobased carbon” are used interchangeably herein. In all embodiments of the present invention, advantageously the retinol is produced by merely organic, renewable, bio-based feedstock, particularly fermentatively produced, as such retinol has an anthropogenic CO₂ emission profile of zero upon biodegradation because all of the CO₂ molecules released during degradation from such “fermentatively-derived” or “fermentatively-produced” retinol have an atmospheric origin. Thus, the net release of CO₂ to the atmosphere is zero.

In one preferred embodiment the mixture of cis and trans isomers of retinol used in the formulations according to the present invention consists essentially of cis and trans isomers of retinol in the ranges and with all the definitions and preferences as given herein. The term “consists essentially of” as used according to the present invention means that the total amount of ingredients within the mixture ideally sum up to 100 wt-%. It is however not excluded that small amounts of impurities or additives may be present in the mixture, with the proviso that the total amount of such impurities is preferably less than about 5 wt-%, more preferably less than about 3, 2, 1 wt-% and which are e.g. introduced via the respective raw materials and/or processes used.

As used herein, the term “impurities” and “additives” are used interchangeably herein and refer to co-ingredients within the inventive mixture and that are present in the inventive mixture in an amount of less than 5 wt-% based on all ingredients present in the mixture. If the mixture according to the present invention consists essentially of retinol with a cis/trans ratio as defined herein, then the purity of said mixture, i.e. the (total) amount of cis and trans retinol, is preferably at least about 95%, more preferably at least about 96, 97, 98 wt-%, most preferably at least about 98%, as determined by known methods such as e.g. HPLC, particularly reversed phase C4 HPLC.

In one embodiment, the mixtures of cis and trans retinol with a cis/trans ratio as defined herein further comprise small amounts of additives such as e.g. dihydro-retinoids, including dihydro-retinol and/or dihydro-retinyl acetate, particularly in a range of 0.2 to 0.01 wt-% or less, such as e.g. 0.2, 0.18, 0.16, 0.15, 0.14, 0.12, 0.1, 0.05, 0.01 wt-% or less, preferably a range of 0.2 to 0.1, 0.17 to 0.06, 0.1 to 0.05, 0.04 to 0.01 wt-%, more preferably a percentage of 0.1 wt.-% or less, all based on total ingredients within said retinol mixture. Also preferably, the percentage of dihydro-retinyl acetate is about 0.05 wt-% or less, particularly about 0.01 wt-% or less based on total ingredients within said retinol mixture.

The present invention relates to a formulation (F4), which is formulation (F), (Fₐ), (F′), (F″), (F″′), (F2), (Fₐ2) or (F3), wherein the comprise small amounts of additives in a range of 0.2 to 0.01 wt-% or less, such as e.g. 0.2, 0.18, 0.16, 0.15, 0.14, 0.12, 0.1, 0.05, 0.01 wt-% or less, preferably a range of 0.2 to 0.1, 0.17 to 0.06, 0.1 to 0.05, 0.04 to 0.01 wt- %, more preferably a percentage of 0.1 wt-% or less, all based on total ingredients within said retinol mixture.

It is obvious that the percentages in all the formulations disclosed in the present patent application are always adding up to 100.

The amount of the retinol in the formulation according to the present invention is 40-75 wt-%, based on the total weight of the formulation.

Preferably the formulation according to the present invention comprises 40-70 wt-%, more preferably 42-70 wt-%, 42-65 wt-%, 45-65 wt-%, 45-60 wt-%, 45-55 wt-%, always based on the total weight of the formulation, of retinol.

Therefore, the present invention relates to a formulation (F5), which is formulation (F),

(Fₐ), (F′), (F″), (F″′), (F1), (Fₐ1), (F2), (Fₐ2), (F3) or (F4) comprising 40-70 wt-%, based on the total weight of the formulation, of retinol.

Therefore, the present invention relates to a formulation (F5′), which is formulation (F), (Fₐ), (F′), (F″), (F″′), (F1), (Fₐ1), (F2), (Fₐ2), (F3) or (F4) comprising 42-70 wt-%, based on the total weight of the formulation, of retinol.

Therefore, the present invention relates to a formulation (F5″), which is formulation (F), (Fₐ), (F′), (F″), (F″′), (F1), (Fₐ1), (F2), (Fₐ2), (F3) or (F4) comprising 42-65 wt-%, based on the total weight of the formulation, of retinol.

Therefore, the present invention relates to a formulation (F5″), which is formulation (F), (Fₐ), (F′), (F″), (F″′), (F1), (Fₐ1), (F2), (Fₐ2), (F3) or (F4) comprising 45-65 wt-%, based on the total weight of the formulation, of retinol.

Therefore, the present invention relates to a formulation (F5″), which is formulation (F), (Fₐ), (F′), (F″), (F″′), (F1), (Fₐ1), (F2), (Fₐ2), (F3) or (F4) comprising 45-60 wt-%, always based on the total weight of the formulation.

Therefore, the present invention relates to a formulation (F5″ ″), which is formulation (F), (Fₐ), (F′), (F″), (F″′), (F1), (Fₐ1), (F2), (Fₐ2), (F3) or (F4) comprising 45-55 wt-%, based on the total weight of the formulation, of retinol.

As stated above the choice of the solvent is crucial for the stability of the formulation according to the present invention.

At least one solvent of formula (I) is used in the formulation according to the present invention.

Preferred solvents are compounds of formula (I)
wherein
R is a

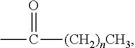

wherein n is an integer chosen from 6, 8 and 14;

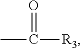

wherein R₃ is a C₁₄-C₂₂ alkene group, and
R₁ is a

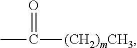

wherein m is an integer chosen from 6, 8 and 14, and
R₂ is a

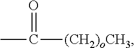

wherein o is an integer chosen from 6, 8 and 14; or

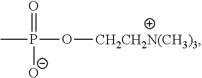

More referred solvents are compounds of formula (Ia) and/or (Ib) as well as a mixture of them

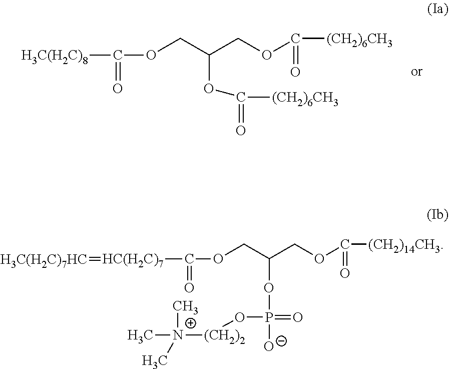

Such suitable solvents are available commercially available from a variety of suppliers (such as i.e. BASF) under tradenames such as i.e. Myritol.

Therefore, the present invention relates to a formulation (F6), which is formulation (F), (Fₐ), (F′), (F″), (F″′), (F1), (Fₐ1), (F2), (Fₐ2), (F3), (F4), (F5), (F5′), (F5″), (F5″′), (F5″″) or (F5″″′), wherein the at least one solvent is a compound of formula (I), wherein
R is a

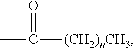

wherein n is an integer chosen from 6, 8 and 14;

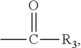

wherein R₃ a C₁₄-C₂₂ alkene group, and
R₁ is a

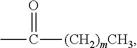

wherein m is an integer chosen from 6, 8 and 14, and
R₂ is a

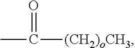

wherein o is an integer chosen from 6, 8 and 14; or

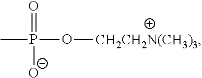

Therefore, the present invention relates to a formulation (F6′), which is formulation (F), (Fₐ), (F′), (F″), (F″′), (F1), (Fₐ1), (F2), (Fₐ2), (F3), (F4), (F5), (F5′), (F5″), (F5″′), (F5″″) or (F5″″′), wherein the at least one solvent is chosen from the group consisting of

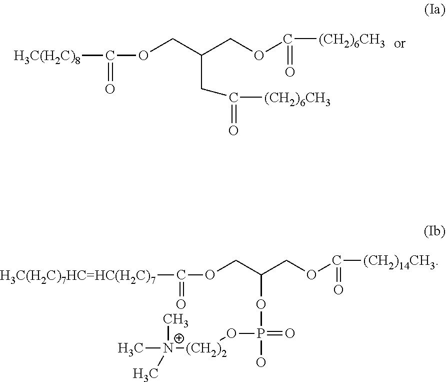

Therefore, the present invention relates to a formulation (F6″), which is formulation (F), (Fₐ), (F′), (F″), (F″′), (F1), (Fₐ1), (F2), (Fₐ2), (F3), (F4), (F5), (F5′), (F5″), (F5″′), (F5″″) or (F5″″′), wherein the solvent is

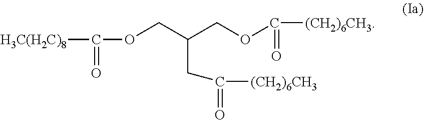

The formulation according to the present invention comprises 20-55 wt-%, based on the total weight of the formulation, of at least one solvent.

Preferably 25-55 wt-%, 28-55 wt-%, 35-55 wt-%, always based on the total weight of the formulation, of at least one solvent.

Therefore the present invention relates to a formulation (F7), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′) or (F6″) comprising 25-55 wt-%, based on the total weight of the formulation, of the at least one solvent.

Therefore the present invention relates to a formulation (F7′), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′) or (F6″) comprising 28-55 wt-%, based on the total weight of the formulation, of the at least one solvent.

Therefore the present invention relates to a formulation (F7″), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′) or (F6″) comprising 35-55 wt-%, based on the total weight of the formulation, of the at least one solvent.

The formulation according to the present invention comprises mixed tocopherols as anti-oxidant.

Mixed tocopherol is a mixture of the following 4 compounds
α-tocopherol and
β-tocopherol and
γ-tocopherol and
δ-tocopherol.

Usually, mixed tocopherol comprises
up to 20 wt-%, based on the total weight of the mixed tocopherol, of α-tocopherol and
up to 5 wt-%, based on the total weight of the mixed tocopherol, of β-tocopherol and
up to 75 wt-%, based on the total weight of the mixed tocopherol, of γ-tocopherol and
up to 35 wt-%, based on the total weight of the mixed tocopherol, of δ-tocopherol.

A preferred mixed tocopherol comprises
10-20 wt-%, based on the total weight of the mixed tocopherol, of α-tocopherol, and
1 to 5 wt-%, based on the total weight of the mixed tocopherol, of β-tocopherol, and
50 to 75 wt-%, based on the total weight of the mixed tocopherol, of γ-tocopherol, and
15 to 35 wt-%, based on the total weight of the mixed tocopherol, of δ-tocopherol.

Therefore, the present invention relates to a formulation (F8), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′) or (F7″), wherein the mixed tocopherol comprises up to 20 wt-%, based on the total weight of the mixed tocopherol, of α-tocopherol, and up to 5 wt-%, based on the total weight of the mixed tocopherol, of β-tocopherol, and up to 75 wt-%, based on the total weight of the mixed tocopherol, of γ-tocopherol, and up to 35 wt-%, based on the total weight of the mixed tocopherol, of 0-tocopherol.

Therefore, the present invention relates to a formulation (F8′), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5′″ ″), (F6), (F6′), (F6″), (F7), (F7′) or (F7″), wherein the mixed tocopherol comprises
10-20 wt-%, based on the total weight of the mixed tocopherol, of α-tocopherol, and
1 to 5 wt-%, based on the total weight of the mixed tocopherol, of β-tocopherol, and
50 to 75 wt-%, based on the total weight of the mixed tocopherol, of γ-tocopherol, and
15 to 35 wt-%, based on the total weight of the mixed tocopherol, of δ-tocopherol.

Mixed tocopherols are commercially available from a variety of suppliers (such as AOM, BASF, DuPont, Merck and DSM).

The formulation according to the present invention does not comprise any further antioxidants (such as BHA and BHT) than the mixed tocopherol.

Therefore, the present invention relates to a formulation (F9), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8) or (F8′), wherein the formulation does not comprise any further antioxidants (other than the mixed tocopherol).

Therefore, the present invention relates to a formulation (F9′), which is formulation (F), (F5″″′), (F6), (F6′), (F6″), (F7), (F7′), (F7″), (F8) or (F8′), wherein the formulation is (essentially) free from BHA and BHT.

The formulations according to the present invention comprises 0.1 to 5 wt-%, based on the total weight of the present invention, of mixed tocopherol.

Preferably, the formulation according to the present invention comprises 0.2 to 4.5 wt-%, 0.2 to 4 wt-%, 0.3 to 4 wt-%, 0.4 to 3.5 wt-%, 0.4 to 3 wt-%, 0.4 to 2.5 wt-%, 0.4 to 2 wt-%, always based on the total weight of the formulation, of mixed tocopherol.

Therefore, the present invention relates to a formulation (F10), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9) or (F9′) comprising 0.2-4.5 wt-%, based on the total weight of the formulation, of mixed tocopherol.

Therefore, the present invention relates to a formulation (F10′), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9) or (F9′) comprising 0.2-4 wt-%, based on the total weight of the formulation, of mixed tocopherol.

Therefore, the present invention relates to a formulation (F10″), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9) or (F9′) comprising 0.3-4 wt-%, based on the total weight of the formulation, of mixed tocopherol.

Therefore, the present invention relates to a formulation (F10″), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9) or (F9′) comprising 0.4-3.5 wt-%, based on the total weight of the formulation, of mixed tocopherol.

Therefore, the present invention relates to a formulation (F10″), which is formulation (F), (F5″″′), (F6), (F6′), (F6″), (F7), (F7′), (F7″), (F8), (F8′), (F9) or (F9′) comprising 0.4-3 wt-%, based on the total weight of the formulation, of mixed tocopherol.

Therefore, the present invention relates to a formulation (F10″), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9) or (F9′) comprising 0.4-2.5 wt-%, based on the total weight of the formulation, of mixed tocopherol.

Therefore, the present invention relates to a formulation (F10″″″), which is formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9) or (F9′) comprising 0.4-2 wt-%, based on the total weight of the formulation, of mixed tocopherol.

The formulation according to the present invention are produced by using commonly known method and using commonly used devices.

A general way to produce the formulation according to the present invention is the following:
Mixing the retinol and the mixed tocopherol at elevated temperature (usually at a temperature range of from 40 to 65° C.).
Heating the at least one solvent up to a similar temperature as the retinol/mixed tocopherol mixture (usually at a temperature range of from 40 to 65° C.).
Adding the at least one solvent to the retinol/mixed tocopherol mixture (or vice versa) at elevated temperature (usually at a temperature range of from 40 to 65° C.) and mixing it at this temperature.
Cool down the mixture slowly.

Therefore, the present invention also relates to the process of producing any of the formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9), (F9′), (F10), (F10′), (F10″), (F10″′), (F10″″), (F10″″′) or (F10″″″) comprising the following steps
Mixing the retinol and the mixed tocopherol at elevated temperature (usually at a temperature range of from 40 to 65° C.)
Heating the at least one solvent up to a similar temperature as the retinol/mixed tocopherol mixture (usually at a temperature range of from 40 to 65° C.).
Adding the at least one solvent to the retinol/mixed tocopherol mixture (or vice versa) at elevated temperature (usually at a temperature range of from 40 to 65° C.) and mixing it at this temperature.
Cool down the mixture slowly.

It is also possible to mix the retinol in the solvent first (usually at a temperature range of from 40 to 65° C.) and then mix it with the mixed tocopherol (usually at a temperature range of from 40 to 65° C.) and the cool down the mixture slowly.

It is also possible to mix the mixed tocopherol in the solvent first (usually at a temperature range of from 40 to 65° C.) and then mix it with the retinol (usually at a temperature range of from 40 to 65° C.) and the cool down the mixture slowly.

The formulations according to the present invention can be used in a variety of fields of application, such as food, feed, pharmaceutical, and personal care.

Preferably the formulations according to the present invention are used for incorporating into personal care products (such as creams, lotions, etc).

Therefore, the present invention also related to the use of at least one formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9), (F9′), (F10), (F10′), (F10″), (F10″′), (F10″″), (F10″″′) or (F10″″″) in food, feed, pharmaceutical, and personal care products.

Therefore, the present invention also related to the use of at least one formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9), (F9′), (F10), (F10′), (F10″), (F10″′), (F10″″), (F10″″′) or (F10″″″) in personal care products (such as creams, lotions).

Furthermore, the present invention also relates to food, feed, pharmaceutical, and personal care products comprising at least one formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9), (F9′), (F10), (F10′), (F10″), (F10″′), (F10″″), (F10″″′) or (F10″″″).

Furthermore, the present invention also relates to personal care products (such as creams, lotions etc) comprising at least one formulation (F), (Fₐ), (F′), (F″), (F″′), (F1), (Fₐ1), (F2), (Fₐ2), (F3), (F4), (F5), (F5′), (F5″), (F5″′), (F5″″), (F5″″′), (F6), (F6′), (F6″), (F7), (F7′), (F7″), (F8), (F8′), (F9), (F9′), (F10), (F10′), (F10″), (F10″′), (F10″″), (F10″″′) or

As stated above the one of the advantages of the formulation according to the present invention is the high amount of retinol (and therefore the reduced amount of other ingredients).

When incorporated into end-market products (food, feed, pharmaceutical, and personal care personal care products) the amount of the formulation depends on how much retinol is needed in these final products.

The following examples serve to illustrate the invention.

### EXAMPLES

All the following examples are made according to the following method: Melting and mixing the retinol and the mixed tocopherol a temperature of 63° C. to 65° C. for about 5 to 10 min under nitrogen;

Then heating the solvent to a temperature of 63° C. to 65° C.

Then mixing (under stirring) the retinol/mixed tocopherol mixture and the solvent at 63° C. to 65° C. for a few minutes 2-5 minutes and the let the formulation cool down to room temperature slowly.

The following solvents have been used are
Mixture of MCT/Lecithin: MCT is medium-chain triglyceride
Myritol 318 (from BASF): this is a Caprylic/Capric Triglyceride

Mixed tocopherols 95 (from DSM) was used

TABLE 1
All values in the table are wt-%, based
on the total weight of the formulation

| Example | 1 | 2 |
|---|---|---|
| Lecithin | — | 21 |
| MCT | — | 30 |
| Myritol 318 | 51 | — |
| Mixed tocopherols 95 | 1 | 1 |
| Retinol (having a cis/trans | 48 | 48 |
| ratio of less than 0.01) | | |

To determine the stability of these formulations, they were stored (at 40° C.) for 2 weeks and 6 weeks. And the loss of retinol was measured (the initial value was 100%).

TABLE 2
the retinol content was determined after storage

| Example | 1 | 2 |
|---|---|---|
| Initial | 100 | 100 |
| 2 weeks | 93.5 | 99 |
| 6 weeks | 88.9 | 93.7 |

It can be seen from the table that the formulations according to the present invention are very stable. The cis/trans ratio of the retinol was also stable.

As stated above, the inventive retinol formulations of the present invention can be incorporated into a variety of compositions.

For example, in the following personal care compositions listed in the following tables (all values are given in weight-%, based on the total weight of the composition)

| Examples: O/W-Emulsions | 3 | 4 | 6 |
|---|---|---|---|
| Cetearyl Alcohol + Sodium | 3.00 | 3.00 | 2.00 |
| Cetylstearylsulfat | | | |
| Glycerylstearat SE | 2.00 | 2.00 | 4.00 |
| Octyldodecanol | 2.00 | 2.00 | 2.00 |
| C₁₂₋₁₅ Alkylbenzoate | 1.00 | 1.00 | 1.00 |
| C13-16 Isoparaffin | 3.00 | 3.00 | 3.00 |
| Undecane, tridecane | 2.00 | 2.00 | 2.00 |
| Glycerine | 5.00 | 5.00 | 6.00 |
| Dimethicone | 0.50 | 0.50 | 0.50 |
| Sodium ascorbylphosphate | 0.10 | | |
| Ethylhexylsalicylate | 0.50 | 0.50 | 0.50 |
| Glycyrrhetic acid | — | — | 0.10 |
| Retinol formulation of Example 1 | 0.1 | 0.16 | 0.02 |
| BEL-EVEN ™ | — | — | 0.50 |
| Grape seed oil | 0.50 | 0.50 | 0.50 |
| Dihydroxyacetone | — | — | 2.00 |
| Paraffinum Liquidum + Ginkgo | 0.25 | 0.25 | 0.25 |
| Biloba Extract | | | |
| Citric acid | 0.09 | 0.09 | 0.09 |
| Sodium citrate | 0.17 | 0.17 | 0.17 |
| Xanthan gum | — | — | 0.10 |
| Ammonium Acryloyldimethyltaurate/ | 0.40 | 0.40 | — |
| VP Copolymer | | | |
| Carbomer | — | — | 0.30 |
| Parabene | 0.30 | 0.30 | 0.30 |
| Phenoxyethanol | 0.50 | 0.50 | 0.50 |
| Alcohol Denat. | 3.50 | 3.50 | 3.50 |
| Perfume | q.s | q.s | q.s |
| Water | ad 100 | ad 100 | ad 100 |

| Examples: O/W-Emulsions | 7 | 8 | 9 |
|---|---|---|---|
| Cetearyl Alcohol + Sodium | 3.00 | 3.00 | 2.00 |
| Cetylstearylsulfat | | | |
| Glycerylstearat SE | 2.00 | 2.00 | 4.00 |
| Octyldodecanol | 2.00 | 2.00 | 2.00 |
| C₁₂₋₁₅ Alkylbenzoate | 1.00 | 1.00 | 1.00 |
| C13-16 Isoparaffin | 3.00 | 3.00 | 3.00 |
| Undecane, tridecane | 2.00 | 2.00 | 2.00 |
| Glycerine | 5.00 | 5.00 | 6.00 |
| Dimethicone | 0.50 | 0.50 | 0.50 |
| Sodium ascorbylphosphate | 0.10 | | |
| Ethylhexylsalicylate | 0.50 | 0.50 | 0.50 |
| Glycyrrhetic acid | — | — | 0.10 |
| Retinol formulation of Example 2 | 0.1 | 0.16 | 0.02 |
| BEL-EVEN ™ | — | — | 0.50 |
| Grape seed oil | 0.50 | 0.50 | 0.50 |
| Dihydroxyacetone | — | — | 2.00 |
| Paraffinum Liquidum + Ginkgo | 0.25 | 0.25 | 0.25 |
| Biloba Extract | | | |
| Citric acid | 0.09 | 0.09 | 0.09 |
| Sodium citrate | 0.17 | 0.17 | 0.17 |
| Xanthan gum | — | — | 0.10 |
| Ammonium Acryloyldimethyltaurate/ | 0.40 | 0.40 | — |
| VP Copolymer | | | |
| Carbomer | — | — | 0.30 |
| Parabene | 0.30 | 0.30 | 0.30 |
| Phenoxyethanol | 0.50 | 0.50 | 0.50 |
| Alcohol Denat. | 3.50 | 3.50 | 3.50 |
| Perfume | q.s | q.s | q.s |
| Water | ad 100 | ad 100 | ad 100 |

| Examples: Hydrodispersions | 10 | 11 | 12 |
|---|---|---|---|
| Glyceryl Stearate Citrate | 0.4 | | |
| Sodium Carbomer | | | |
| Acrylates/C₁₀₋₃₀ Alkyl Acrylate | | 0.1 | 0.2 |
| Crosspolymer | | | |
| Ceteareth-20 | | | |
| Potassium Cetyl Phosphate | | | |
| Xanthan Gum | | 0.5 | 0.2 |
| Dimethicone/Vinyl Dimethicone | | 3 | 1.5 |
| Crosspolymer | | | |
| 2-(4-Diethylamino-2-hydroxybenzoyl)- | | 1.5 | |
| benzoic Acid Hexylester | | | |
| 2,4,6-Tribiphenyl-4-yl-1,3,5 Triazine | | | 0.5 |
| Butyl Methoxydibenzoylmethane | | | 5 |
| Ethylhexyl Bis-Isopentylbenzoxazolyl- | | 2 | |
| phenyl Melamine | | | |
| Bis-Ethylhexyloxyphenol Methoxy- | 2 | | |
| phenyltriazine | | | |
| Disodium Phenyl Dibenzimidazole | | 1 | |
| Tetrasulfonate | | | |
| Phenylbenzimidazole Sulfonic Acid | 1 | | |
| Ethylhexyl Methoxycinnamate | | 8 | |
| Ethylhexyl Salicylate | | | |
| Homosalate | | | |
| Diethylhexyl Butamido Triazone | | | |
| Ethylhexyl Triazone | 3 | | 1 |
| Octocrylene | | | |
| Polysilicone-15 | 0.9 | | |
| Methylbenzylidene Camphor | | | 2 |
| BEL-EVEN ™ | 0.5 | — | — |
| Titanium Dioxide | 2 | 1 | 2 |
| Drometrizole Trisiloxane | | 0.5 | |
| Terephthalidene Dicamphor Sulfonic Acid | | 0.75 | |
| C₁₂₋₁₅ Alkyl Benzoate | | | |
| Butylene Glycol Dicaprylate/Dicaprate | | | |
| Dicaprylyl Carbonate | 3 | | |
| Dicaprylylether | 2 | | |
| Cyclomethicone | | | 3 |
| 2-Phenylethylbenzoate | | | |
| Diethylhexylnaphthalate | | | |
| Tridecylsalicylate | 3 | 0.5 | 3 |
| PVP Hexadecene Copolymer | | 1 | |
| Glycerin | 5 | 8 | |
| Butylene Glycol | 7 | | |
| Glycine Soja | | | 1 |
| Vitamin E Acetate | 0.25 | 1 | 0.25 |
| Alpha-Glycosylrutin | | | |
| Retinol formulation of Example 1 | 0.1 | 0.02 | 0.1 |
| DHA | | 1.0 | |
| Erythrulose | | 0.5 | |
| Trisodium EDTA | 0.1 | | 0.1 |
| Tromethamine | | | |
| Ethanol | 10 | 1 | |
| Preservatives | q.s | q.s | q.s |
| Fragrance, Colours | q.s | q.s | q.s |
| Water | Ad 100 | Ad 100 | Ad 100 |

| Examples: Hydrodispersions | 13 | 14 | 15 |
|---|---|---|---|
| Glyceryl Stearate Citrate | 0.4 | | |
| Sodium Carbomer | | | |
| Acrylates/C₁₀₋₃₀ Alkyl Acrylate | | 0.1 | 0.2 |
| Crosspolymer | | | |
| Ceteareth-20 | | | |
| Potassium Cetyl Phosphate | | | |
| Xanthan Gum | | 0.5 | 0.2 |
| Dimethicone/Vinyl Dimethicone | | 3 | 1.5 |
| Crosspolymer | | | |
| 2-(4-Diethylamino-2-hydroxybenzoyl)- | | 1.5 | |
| benzoic Acid Hexylester | | | |
| 2,4,6-Tribiphenyl-4-yl-1,3,5 Triazine | | | 0.5 |
| Butyl Methoxydibenzoylmethane | | | 5 |
| Ethylhexyl Bis-Isopentylbenzoxazolyl- | | 2 | |
| phenyl Melamine | | | |
| Bis-Ethylhexyloxyphenol Methoxy- | 2 | | |
| phenyltriazine | | | |
| Disodium Phenyl Dibenzimidazole | | 1 | |
| Tetrasulfonate | | | |
| Phenylbenzimidazole Sulfonic Acid | 1 | | |
| Ethylhexyl Methoxycinnamate | | 8 | |
| Ethylhexyl Salicylate | | | |
| Homosalate | | | |
| Diethylhexyl Butamido Triazone | | | |
| Ethylhexyl Triazone | 3 | | 1 |
| Octocrylene | | | |
| Polysilicone-15 | 0.9 | | |
| Methylbenzylidene Camphor | | | 2 |
| BEL-EVEN ™ | 0.5 | — | — |
| Titanium Dioxide | 2 | 1 | 2 |
| Drometrizole Trisiloxane | | 0.5 | |
| Terephthalidene Dicamphor Sulfonic Acid | | 0.75 | |
| C₁₂₋₁₅ Alkyl Benzoate | | | |
| Butylene Glycol Dicaprylate/Dicaprate | | | |
| Dicaprylyl Carbonate | 3 | | |
| Dicaprylylether | 2 | | |
| Cyclomethicone | | | 3 |
| 2-Phenylethylbenzoate | | | |
| Diethylhexylnaphthalate | | | |
| Tridecylsalicylate | 3 | 0.5 | 3 |
| PVP Hexadecene Copolymer | | 1 | |
| Glycerin | 5 | 8 | |
| Butylene Glycol | 7 | | |
| Glycine Soja | | | 1 |
| Vitamin E Acetate | 0.25 | 1 | 0.25 |
| Alpha-Glycosylrutin | | | |
| Retinol formulation of Example 2 | 0.1 | 0.02 | 0.1 |
| DHA | | 1.0 | |
| Erythrulose | | 0.5 | |
| Trisodium EDTA | 0.1 | | 0.1 |
| Tromethamine | | | |
| Ethanol | 10 | 1 | |
| Preservatives | q.s | q.s | q.s |
| Fragrance, Colours | q.s | q.s | q.s |
| Water | Ad 100 | Ad 100 | Ad 100 |

1. A formulation comprising
40-75 weight-% (wt-%), based on the total weight of the formulation, of retinol, and
20-55 wt-%, based on the total weight of the formulation, of at least one solvent, and
0.1-5 wt-%, based on the total weight of the formulation, of mixed tocopherol,
wherein the at least one solvent is a compound of formula (I) 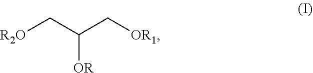
wherein
R is a 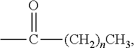
wherein n is an integer from 4 to 16; 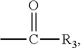
wherein R₃ is a C₁₄-C₂₂ alkene group, and
R₁ is a 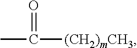
wherein m is an integer from 4 to 16, and
R₂ is a 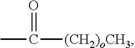
wherein o is an integer from 4 to 16; or 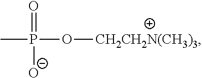
characterised in that
the retinol is a mixture of cis and trans isomers wherein
the cis/trans ratio within said retinol mixture is less than 0.01.

2. A formulation consisting (essentially) of
40-75 wt-%, based on the total weight of the formulation, of retinol, and
20-55 wt-%, based on the total weight of the formulation, of at least one solvent, and
0.1-5 wt-%, based on the total weight of the formulation, of mixed tocopherol,
wherein the at least one solvent is a compound of formula (I) 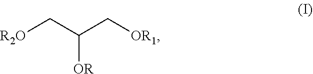
wherein
R is a 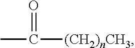
wherein n is an integer from 4 to 16; 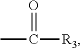
wherein R₃ is a C₁₄-C₂₂ alkene group, and
R₁ is a 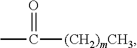
wherein m is an integer from 4 to 16, and
R₂ is a 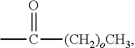
wherein o is an integer from 4 to 16; or 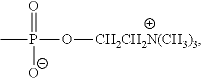
characterised in that
the retinol is a mixture of cis and trans isomers wherein
the cis/trans ratio within said retinol mixture is less than 0.01.

3. The formulation according to claim 1, wherein retinol is biologically produced through a fermentation process.

4. The formulation according to claim 1 comprising 40-70 wt-%, based on the total weight of the formulation, of retinol.

5. The formulation according to claim 1, wherein the at least one solvent is a compound of formula (I),
wherein
R is a 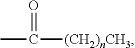
wherein n is an integer chosen from 6, 8 and 14; 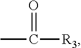
wherein R₃ is a C₁₄-C₂₂ alkene group, and
R₁ is a 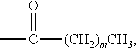
wherein m is an integer chosen from 6, 8 and 14, and
R₂ is a 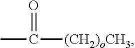
wherein o is an integer chosen from 6, 8 and 14; or

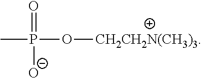

6. The formulation according to claim 1, wherein the solvent is chosen from the group consisting of

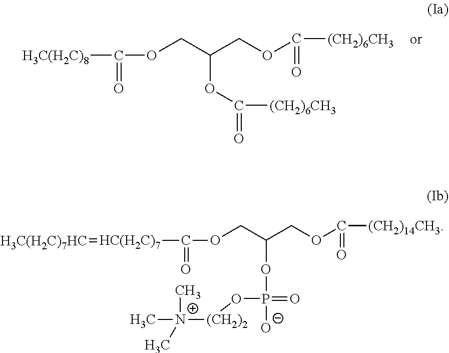

7. The formulation according to claim 1, comprising 25-55 wt-%, based on the total weight of the formulation, of the at least one solvent.

8. The formulation according to claim 1, wherein the mixed tocopherol comprises
up to 20 wt-%, based on the total weight of the mixed tocopherol, of α-tocopherol, and
up to 5 wt-%, based on the total weight of the mixed tocopherol, of β-tocopherol, and
up to 75 wt-%, based on the total weight of the mixed tocopherol, of γ-tocopherol, and
up to 35 wt-%, based on the total weight of the mixed tocopherol, of δ-tocopherol.

9. The formulation according to claim 1, wherein the formulation does not comprise any further antioxidants (other than the mixed tocopherol).

10. The formulation according to claim 1, comprising 0.2-4.5 wt-%, based on the total weight of the formulation, of mixed tocopherol.

11. Process of producing a formulation according to claim 1 comprising the following steps
Mixing the retinol and the mixed tocopherol at elevated temperature (at a temperature range of from 40 to 65° C.)
Heating the at least one solvent up to a similar temperature as the retinol/mixed tocopherol mixture (40 to 65° C.)
Adding the at least one solvent to the retinol/mixed tocopherol mixture (or vice versa) at elevated temperature (at a temperature range of from 40 to 65° C.) and mixing it at this temperature
Cool down the mixture slowly.

12. Use of at least one formulation according to claim 1 in food, feed, pharmaceutical, and personal care products (preferably in personal care products).

13. Food, feed, pharmaceutical, and personal care products comprising at least one formulation according to claim 1.

14. Personal care products comprising at least one formulation according to claim 1.
